(19) United States
(12) Patent Application Publication
(10) Pub. No.: US 2015/0290417 A1
(43) Pub. Date: Oct. 15, 2015

(54) System and Method for High Concentration Nitric Oxide Delivery

(71) Applicant: 12th Man Technologies. Inc., Garden Grove, CA (US)

(72) Inventors: Alex Stenzler, Long Beach, CA (US); Christopher C. Miller, North Vancouver (CA); Steve Han, Huntington Beach, CA (US)

(21) Appl. No.: 14/682,698

(22) Filed: Apr. 9, 2015

Related U.S. Application Data
(60) Provisional application No. 61/977,448, filed on Apr. 9, 2014.

(51) Int. Cl. A61M 16/12; A61K 33/00; A61M 16/06; A61K 9/00

(52) U.S. Cl. CPC A61M 16/12; A61K 9/007; A61K 33/00; A61M 16/06

(57) ABSTRACT

The present invention relates to systems and methods for administering high concentrations of nitric oxide (NO) gas to a patient without the need to provide supplemental oxygen to the patient. The systems and methods can be used to administer high therapeutic amounts of NO gas, for example a gas comprising 160 ppm NO, while forming little or no residual nitrogen dioxide (NO₂). The method is based on using a NO gas source with a relatively high NO concentration, for example 5,000 ppm, while rapidly mixing the gas from the NO gas source with air immediately prior to administering the gas to a patient.

### CROSS-REFERENCE TO RELATED APPLICATIONS

This application claims priority to U.S. Patent Application Ser. No. 61/977,448, filed Apr. 9, 2014, the entire contents of which are incorporated herein by reference.

### BACKGROUND OF THE INVENTION

The administration of nitric oxide (NO) gas can be used for various therapeutic applications. For example, NO is currently approved for use in the U.S. to treat pulmonary hypertension in newborns. Typically, NO is delivered to patients while they are on mechanical ventilators, and the NO is injected into the breathing circuit before the inspired gas enters the humidifier of the ventilator. Systems for injecting NO typically monitor the patient's dynamic inspiratory flow, and inject NO proportionally to the inspired flow to keep the concentration of NO in the inhaled gas constant (see, e.g., Bathe et al., U.S. Pat. No. 5,558,083 and Bathe et al., U.S. Pat. No. 6,125,846, the contents of both of which are incorporated by reference in their entireties). However, there can be a long, more than a second, contact period between NO and oxygen in such systems because the NO gas is often injected into the ventilator circuit a substantial distance, perhaps up to six feet, upstream from the point where it enters the patient's lungs. A long contact period can be problematic because NO and oxygen react to form nitrogen dioxide (NO₂), which is highly toxic and must be kept below about 3-5 ppm in the air inhaled by the patient. The National Institute of Occupational Safety and Health's time weighted average requires that inhaled NO₂ be less than 5 ppm in the work environment, and the US Food and Drug Administration has even lower requirements for patients.

In current therapeutic use, NO is typically administered at a concentration of about 20 ppm. The NO source gas used for such therapeutic use is typically stored in cylinders with a NO concentration in the range of 400 to 1,000 ppm (0.04 to 0.1%). The remainder of the source gas is generally nitrogen, so that the NO does not get exposed to oxygen while it is being stored prior to use. When NO is administered to a patient at a concentration of 20 ppm, the oxygen content of the inspired air after being mixed with the NO source gas is generally maintained at a safe level, albeit at a slightly lower percentage. For example, using a source gas with a NO concentration of 1,000 ppm, the oxygen content of the inspired air will only be reduced by about 2%. In such an example, the inspired oxygen would be reduced from about 21% to 20.6%. This is approximately the equivalent of standing on the 35th floor of an office building at sea level, and poses no risk to the patient.

However, other potential therapeutic uses of NO will likely benefit from NO being administered at concentrations higher than 20 ppm. For example, a 160 ppm NO concentration would be useful for antimicrobial applications. In such an example, administering NO using a 1,000 ppm source gas would reduce the oxygen concentration in the inspired air, after mixing with the source gas, to about 17%. Such a reduction in oxygen would require the addition of supplemental oxygen to prevent hypoxemia. However, the addition of supplemental oxygen is undesirable because it requires additional equipment, including an oxygen source, and increases the risk of NO₂ formation because higher oxygen concentrations increases the reaction rate between NO and oxygen.

Thus, there is a continuing need in the art for systems and methods to deliver a high therapeutic concentration of nitric oxide gas to a patient while minimizing the formation of NO₂ and avoiding the need for adding supplemental oxygen. The present invention addresses this continuing need in the art.

### BRIEF DESCRIPTION OF THE DRAWINGS

The features and advantages of the present invention will be more fully understood with reference to the following, detailed description when taken in conjunction with the accompanying figures, wherein:

FIG. 1 is a block diagram representing an exemplary embodiment of a system of the present invention;

FIG. 2 is an image of an exemplary embodiment of a system of the present invention;

FIG. 3 is a schematic diagram of another exemplary embodiment of a system of the present invention;

FIG. 4 is a cross-sectional schematic diagram of an exemplary embodiment of a gas mixing device useful in a system of the present invention;

FIG. 5 is a schematic diagram of an exemplary embodiment of a gas mixing swirl plate useful in a system of the present invention;

FIG. 6 is a block diagram of an exemplary embodiment of a method of the present invention;

FIG. 7 is a block diagram of another exemplary embodiment of a method of the present invention;

FIG. 8 is a block diagram of yet another exemplary embodiment of a method of the present invention; and

FIG. 9 is a graph showing data acquired during a test run of an embodiment of a system of the present invention. Nitric oxide (left y-axis values) was maintained at about 160 ppm while the NO₂ concentration (right y-axis values) was held at about 3 ppm or less. The concentrations were measured at 1 second intervals (x-axis values are in seconds). The solid line represents flow measurements, wherein each “bump” on the graph corresponds to a simulated breath.

### DETAILED DESCRIPTION

It is to be understood that the figures and descriptions of the present invention have been simplified to illustrate elements that are relevant for a clear understanding of the present invention, while eliminating, for the purpose of clarity, many other elements found in typical gas delivery systems or methods, or therapeutic gas administration methods. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is, at times, not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

An “effective amount” or “therapeutically effective amount” of a compound is that amount of compound which is sufficient to provide a beneficial effect to the subject to which the compound is administered and which exits the system after being blended.

The terms “patient,” “subject,” “individual,” and the like are used interchangeably herein, and refer to any animal amenable to the methods described herein. In certain non-limiting embodiments, the patient, subject or individual is a livestock animal, such as cattle, or it may be a human.

A “therapeutic” treatment is a treatment administered to a subject who exhibits signs and/or symptoms of a disease or disorder, for the purpose of diminishing or eliminating those signs and/or symptoms.

Diseases or disorders that may be amenable to such therapeutic treatments include but are not limited to cystic fibrosis, tuberculosis, bronchiolitis, pneumonia, bronchiectasis, bronchitis, influenza, respiratory syncytial virus, sinusitus, tracheitis, upper respiratory tract infection, and lower respiratory tract infection.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, 6 and any whole and partial increments therebetween. This applies regardless of the breadth of the range.

### DESCRIPTION

The present invention relates to systems and methods for using high concentrations of nitric oxide (NO) greater than 3000 ppm to administer high concentration therapeutic NO gas to a patient without the need to provide supplemental oxygen to the patient. The systems and methods can be used to administer high therapeutic amounts of NO gas (e.g., 100-400 ppm NO, 160 ppm NO, etc.) while forming little or no residual nitrogen dioxide (NO₂). The systems and methods are based on using a NO gas source with a relatively high NO concentration (e.g., 3,000 to 10,000 ppm NO, 5000 ppm NO, etc.), while rapidly mixing the gas from the NO gas source with air immediately prior to administering the gas to a patient. Rapid mixing of gas is required for the present invention so that NO gas can be injected into the device only a short distance from the entrance to the patient's airway at their nose or mouth, thereby reducing the potential contact time for the NO gas to be exposed to oxygen. By reducing the potential contact time between NO gas and oxygen, the conversion of NO gas to highly toxic NO₂ gas can be reduced or eliminated. Accordingly, systems and methods of the present invention can rapidly mix gases at a position in a gas administration system that is close to the point of inhalation by the patient.

Systems and methods of the present invention can be used for administering a higher concentration of NO gas for therapeutic use than treatment methods currently used in the art (i.e., NO concentrations significantly higher than 20 ppm). In various embodiments, the concentration of NO in the gas delivered to the patient (i.e., the delivery gas, desired therapeutic concentration, concentration of NO mixed when mixed with air and/or O2, etc.) can be any concentration in the range of about 100-400 ppm. In exemplary embodiments, the concentration of NO in the delivery gas is about 160 ppm.

High concentrations of inhaled NO for therapeutic applications can be difficult to administer because of the reduction of inspired oxygen from the high percentage of nitrogen in the inspired gas, and can be potentially dangerous to the patient because of the potential for NO₂ formation. As mentioned previously, NO is unstable in the presence of oxygen because it converts to NO₂, and, therefore, NO must be stored in a relatively oxygen-free carrier gas, typically nitrogen, which is then blended with air or oxygen into a breathing circuit prior to inhalation by the patient. The nitrogen added into the breathing circuit as part of the NO source gas reduces the oxygen in the delivery gas, which can require the replacement of oxygen in the delivery gas if the oxygen concentration decreases below 20 percent. However, high local concentrations of oxygen can result in the rapid conversion of NO to NO₂ as the concentration of oxygen directly affects the conversion rate of the reaction with an approximately five-fold faster conversion in 100% oxygen as compared with 21% oxygen [M. Francoe, E. Troncy, and G. Blaise. Inhaled nitric oxide: Technical aspects of administration and monitoring. Crt Care Med 1998. vol 26. 4:782-794]. In addition, the use of nearly pure (˜100%) NO gas as a NO source to delivery 160 ppm (0.016%) is undesirable because it can create difficulty in controlling a consistent concentration of a very low percentage of NO in the delivery gas. When using nearly pure NO gas, rapid fluctuations of NO in the delivery gas can occur due to reduced precision in control, which can result in either an unsafe level of NO gas or an ineffective amount of NO gas administered to the patient or sudden dangerous spikes in NO₂ formation. Patients do not breathe at a constant flow. The inspiratory flow of a patient can vary by more than 200 fold during a period of less than 1 second as they breathe in and a mixing system would need to respond precisely and as quickly, a difficult task with the known art. As an example, to use pure NO to deliver 200 ppm of NO, it would require dynamic precision flow at 0.02% of the patient's inspiratory flow while most flow sensors are limited to precision of 0.1% under dynamic conditions.

Systems and methods of the present invention solve at least these problems by providing the ability to safely maintain the desired dosage level of NO in the delivery gas, while also providing both a level of oxygen suitable for breathing and an acceptably low level of NO₂ in the delivery gas. In at least some embodiments, NO source gas with a relatively high NO concentration is mixed with air to generate the delivery gas. By using a NO source gas with a relatively high concentration of NO, the need for adding supplemental oxygen to the delivery gas to maintain an acceptable level of oxygen in the delivery gas can be eliminated. In at least one embodiments, the source gas can be in the range of about 3,000 to 10,000 ppm NO. In exemplary embodiments, the source gas has a concentration of about 5,000 ppm NO. The NO source gas is typically provided in a cylinder that can be readily connected to a gas administration device, however, the source gas can be provided in any type of container as would be understood by a person skilled in the art and/or the NO source gas can be provided from an NO generator, chemical reaction, and/or any NO source as would be understood by a person skilled in the art.

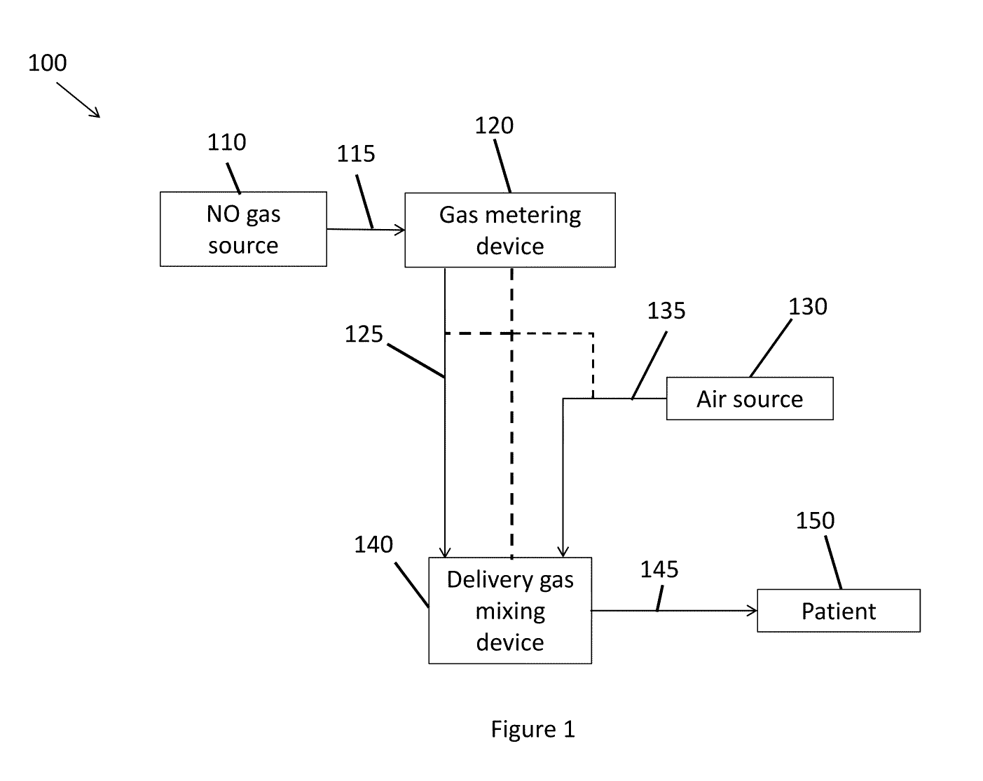

Referring now to FIG. 1, a diagram of an exemplary therapeutic gas system 100 of the present invention is shown. System 100 comprises a NO gas source 110, which is connected to and/or in fluid communication with a gas metering device 120 via a conduit 115. In at least one embodiment, NO gas source 110 is a cylinder having a NO concentration in the range of 3,000 to 10,000 ppm. In another embodiment, the NO concentration in the cylinder is about 5,000 ppm. NO gas can flow from NO gas source 110 via conduit 115 to metering device 120. The NO gas can then be released from metering device 120 in a controlled fashion to a delivery gas mixing and administration device 140 via a conduit 125. Air can also be transferred to the delivery gas mixing and administration device 140 from an air source 130 via conduit 135. In at least some embodiments, air source 130 is ambient air. In at least one embodiment, air source 130 can be an air cylinder and/or the air source can be any pressurized source of air. The NO gas transferred via conduit 125 is mixed with air from air source 130 in mixing and administration device 140 to generate a delivery gas, which is then delivered to the patient 150 via a patient interface 145. Mixing and administration device 140 can use one of many gas mixer elements described in the art, for example the device described by Skimming et al. (U.S. Pat. No. 5,722,392). Further, metering device 120 can be used to control the amount of NO gas being supplied to mixing device 140, so that the delivery gas entering patient interface 145 meets predetermined composition requirements.

In various embodiments, the delivery gas inhaled via patient interface 145 comprises a therapeutic concentration of NO gas, a concentration of oxygen in an acceptable range for the patient, and either little or no NO₂. In at least one embodiment, the concentration of NO gas is in the range of 100 to 400 ppm NO and the oxygen concentration is at least about 20%. In various embodiments, the NO₂ concentration can be less than 5 ppm, less than 3 ppm, less than 1 ppm, or any other concentration that would be considered safe and suitably low, as would be understood by a person skilled in the art.

In at least one embodiment, the air from the air source flows through the gas mixing device at a predetermined, relatively constant flow rate, and the NO source gas is mixed into the air at a relatively constant flow rate, based on a predetermined setting and/or theoretical calculation, so that the NO concentration in the resulting delivery gas remains at the desired concentration. In at least some embodiments, metering device 120 measures the flow of air flowing through the device and meters NO source gas at a rate proportional to the flow rate of air, so that the concentration of NO in the delivery gas remains constant at the desired concentration. While in the first described embodiment, there are no dynamic fluctuations in flow that require rapid flow matching of the injected NO, the volume of gas must be very high to meet the highest inspiratory flow of the patient, thereby wasting gas at periods of lower inspiratory flow. In the second embodiment, there is no waste of NO gas, but the requirements for rapid and accurate mixing are critical. Metering devices capable of dynamically metering are known in the art, for example the devices described by Bathe et al., U.S. Pat. No. 5,558,083 and/or Figley et al., U.S. Pat. No. 6,955,171 the contents of both of which are incorporated by reference in their entireties.

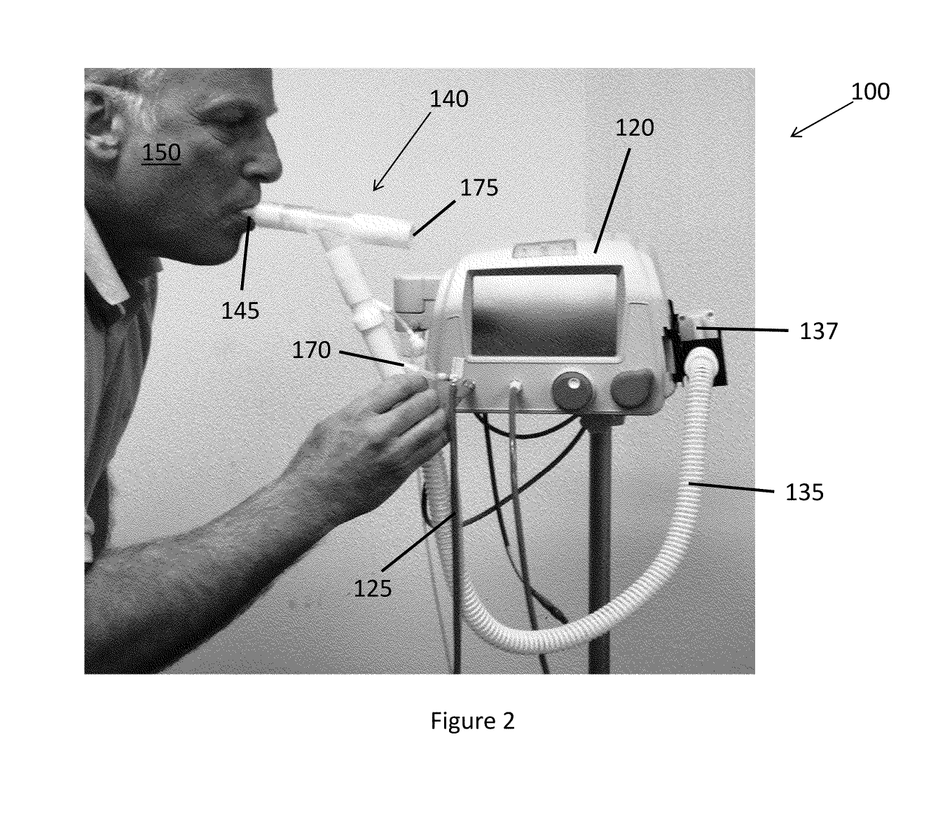

Referring to FIG. 2, another exemplary embodiment of system 100 is shown. Ambient air from the surrounding environment is transferred to and/or received by mixing and administration device 140 via conduit 135. The flow rate of air flowing through conduit 135 is measured using a flow meter 137. An NO gas source (not shown) is connected to and/or in fluid communication with metering device 120. NO gas is supplied to metering device 120, then released from metering device 120 to and/or received by mixing and administration device 140 via conduit 125 in proportion to the flow rate of air measured. The air and NO gas is mixed within mixing and administration device 140, and patient 150 inhales the mixed delivery gas from mixing and administration device 140 via patient interface 145. The patient can then exhale through outlet 175 in mixing and administration device 140. In at least one embodiment, patient interface 145 is a breathing tube that is incorporated into mixing and administration device 140 for inhalation via the patient's mouth. Of course patient interface 145 can be a gas delivery mask, or any other mechanism for administering gases to a patient known in the art. As shown in FIG. 2, the NO gas delivered via conduit 125 enters mixing and administration device 140 via a conduit 170 that is a relatively short distance from where the delivery gas can exit patient interface 145 and enter the patient. In at least one embodiment, mixing and administration device 140 comprises a swirl plate that is used to rapidly mix the NO gas and air to form the delivery gas just prior to the delivery gas exiting mixing and administration device 140.

Accordingly, the relatively short distance between where the NO gas enters and exits mixing and administration device 140 allows only a short amount of time for the NO gas to mix with the oxygen in the air. Therefore, the potential for the creation of harmful NO₂ gas is greatly minimized or even eliminated. In one embodiment, the distance between where the NO gas enters and exits mixing and administration device 140 is about 6 inches. In another embodiment, the distance is about 4 inches. In yet another embodiment, the distance is about 2 inches. However, the distance between where the NO gas enters and exits mixing and administration device 140 can be any distance that is suitable short to minimize or prevent NO₂ gas formation while allowing for sufficient mixing of NO gas and air to provide a delivery gas with a relatively uniform concentration.

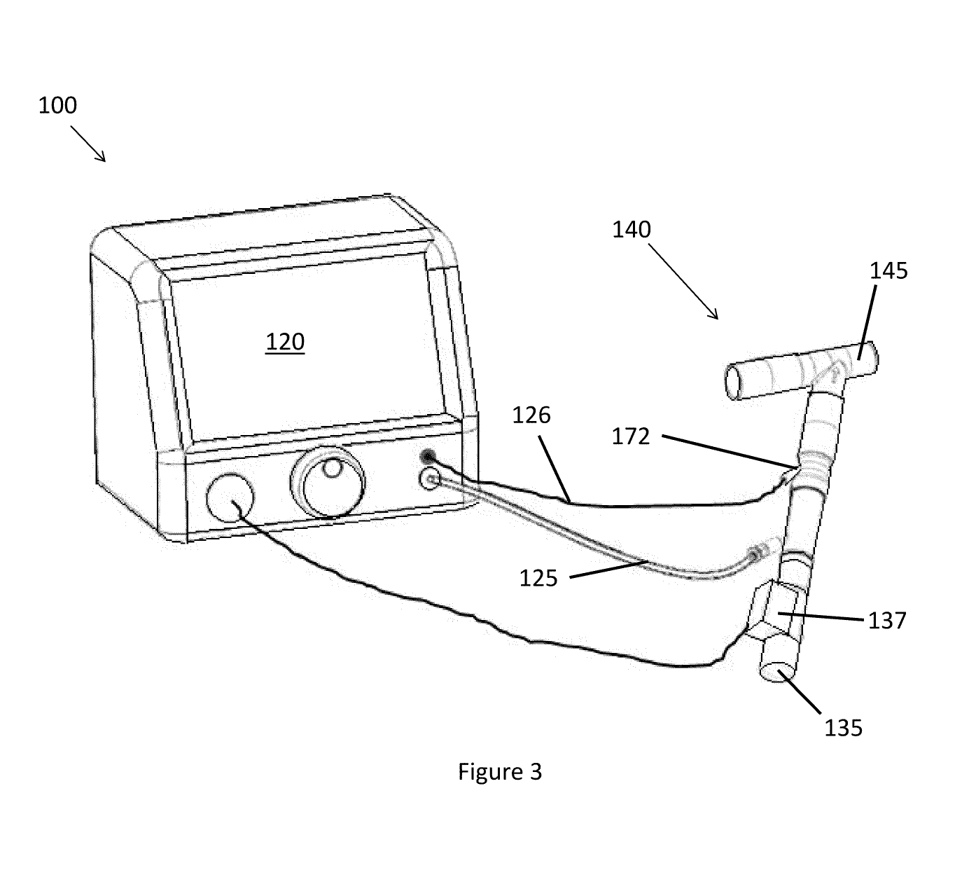

Referring to FIG. 3, another embodiment of system 100 is shown. In at least one embodiments, air is drawn into mixing and administration device 140 via a conduit 135 that is open to the surrounding atmosphere, where the flow of air flow rate is measured by a flow sensor 137. NO gas is released to and/or received by mixing and administration device 140 via conduit 125 connected to metering device 120, wherein the NO gas is proportionally mixed with the air flowing through the mixing device.

In exemplary embodiments, metering device 120 can be connected to, coupled to, and/or in fluid communication with a port 172 on mixing and administration device 140, via conduit 126, that can be used to sample gas flowing through mixing and administration device 140, so that the concentration of NO gas in the delivery gas can be measured.

In exemplary embodiments, systems and methods of the present invention can include a sensor 137 for measuring the flow of air entering mixing and administration device 140. Sensor 137 can be connected to metering device 120 via wires (as shown) or via any wireless connection, such as BLUETOOTH or WiFi. Accordingly, metering device 120 can be used to control the concentration of NO in the delivery gas exiting patient interface 145 by measuring the flow of air and/or the concentration of NO after the NO source gas is mixed with air, and by adjusting the flow of NO source gas transferred via conduit 125 to mixing and administration device 140.

In various embodiments of the present invention, metering device 120 can be used to control the flow of NO gas into mixing and administration device 140. Accordingly, metering device 120 can be used to control the concentration of NO in the delivery gas being administered via patient interface 145. Metering device 120 can further comprise components necessary for controlling the flow of gases and/or for adjusting the concentration of NO gas in the delivery gas, including, but not limited to: one or more sensors for measuring the concentration of one or more components, for example NO₂, NO, or oxygen, of the delivery gas, NO source gas, or air source; one or more sensors for measuring the flow of the delivery gas, NO source gas, or air source; one or more sensors for measuring other parameters of the system, for example pressure; one or more valves for controlling the flow of gas in the system 100; a microprocessor or logic circuit for receiving data signals from the one or more sensors, analyzing such data signals, calculating parameters related to controlling the concentration of NO in the delivery gas, and/or sending signals to the one or more valves in system 100; a user input mechanism; a display mechanism; and an alarm mechanism. In one embodiment, the metering device can be any type of gas delivery system known in the art, for example the systems described by Bathe et al. (U.S. Pat. No. 5,558,083) or Figley et al. (U.S. Pat. No. 6,955,171).

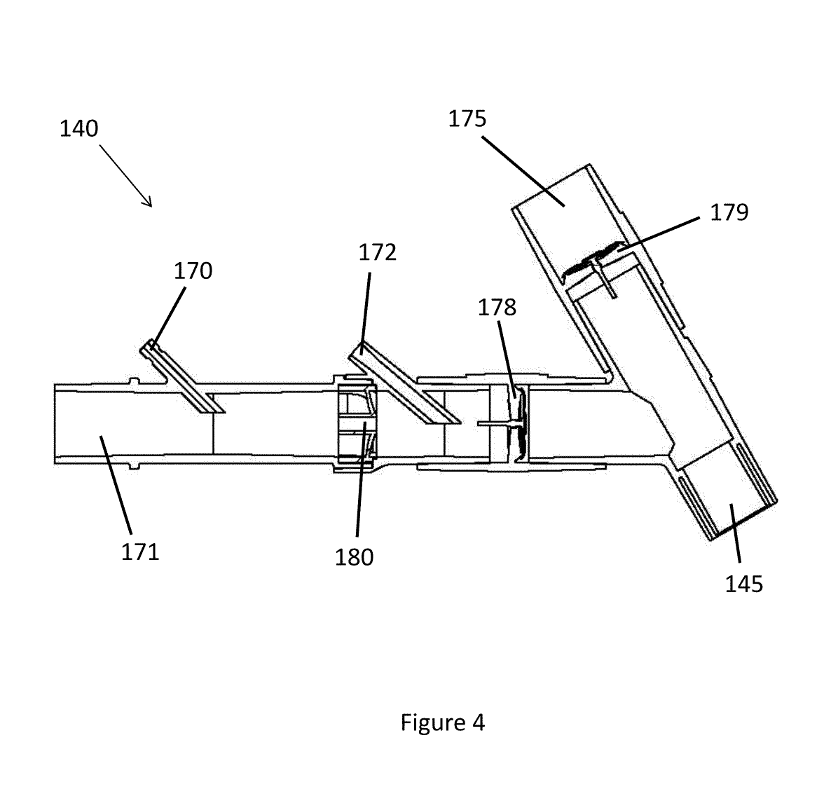

Referring now to FIG. 4, a cross-sectional diagram of an exemplary embodiment of gas mixing and administration device 140 is shown. Device 140 has a conduit 170 for transferring and/or receiving NO source gas and a conduit 171 for transferring and/or receiving air into the device. The mixing of the NO source gas and air is promoted by a swirl plate 180 to rapidly mix the gases and to generate a substantially uniform delivery gas having the desired NO concentration. The concentration, or some other property of the delivery gas, can be measured by inserting a sample line from a sensor or transducer into port 172, or by placing a sensor directly in device 140. The delivery gas then exits device 140 via a patient interface 145 (e.g., a portion of the device configured in the shape of a tube suitable for interfacing with a patient's mouth). Device 140 can further comprise an exhaust port and conduit 175 for allowing gas exhaled by the patient to vent to the surrounding environment. Accordingly, device 140 can also comprise one or more check valves 178 and 179 for directing the flow of inhaled gas to the patient and exhaled gas through conduit 175 and into the surrounding environment without allowing the exhaled air to mix with the delivery gas. The resistance to air flow of these valves is less than 5 cmH₂O at 50 liters per minute so that there is no significant increase in the work of breathing for the patients being treated. In an exemplary embodiment, the resistance to air flow is less than 2 cmH₂O at 50 liters per minute.

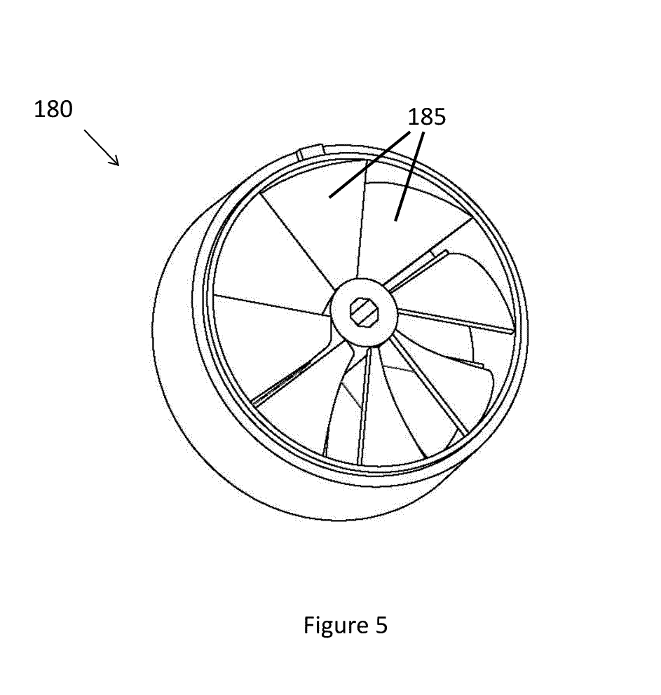

Referring now to FIG. 5, a perspective view of swirl plate 180 is shown. Swirl plate 180 comprises a number of blades 185. When gases pass through the blades, the flow pattern of the gases is deflected, thereby causing rapid mixing of the gases.

In exemplary embodiments, systems and methods of the present invention administer a relatively high dose of NO gas to a patient. The methods described herein may be suitable for treating diseases or disorders such as, and without limitation, cystic fibrosis, tuberculosis, bronchiolitis, pneumonia, bronchiectasis, bronchitis, influenza, respiratory syncytial virus, sinusitus, tracheitis, upper respiratory tract infection, and lower respiratory tract infection.

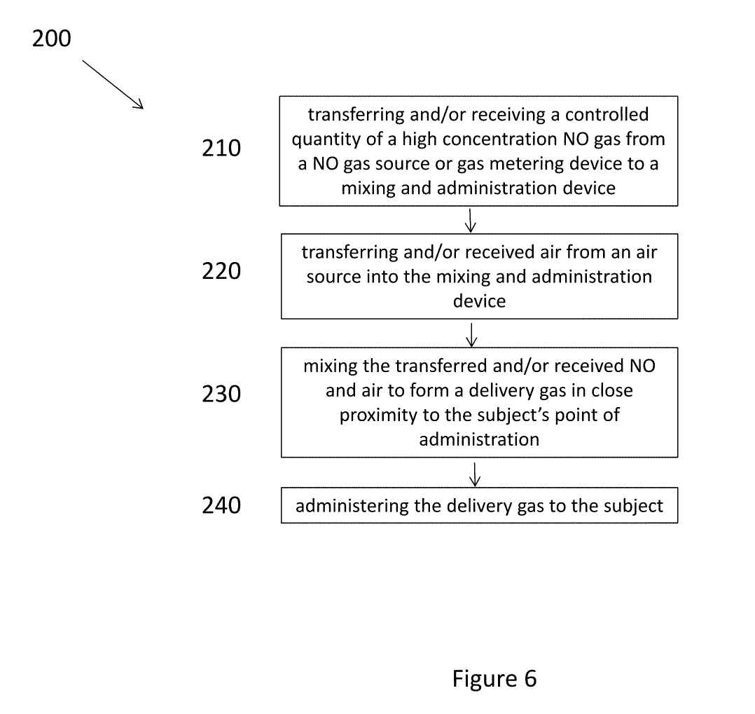

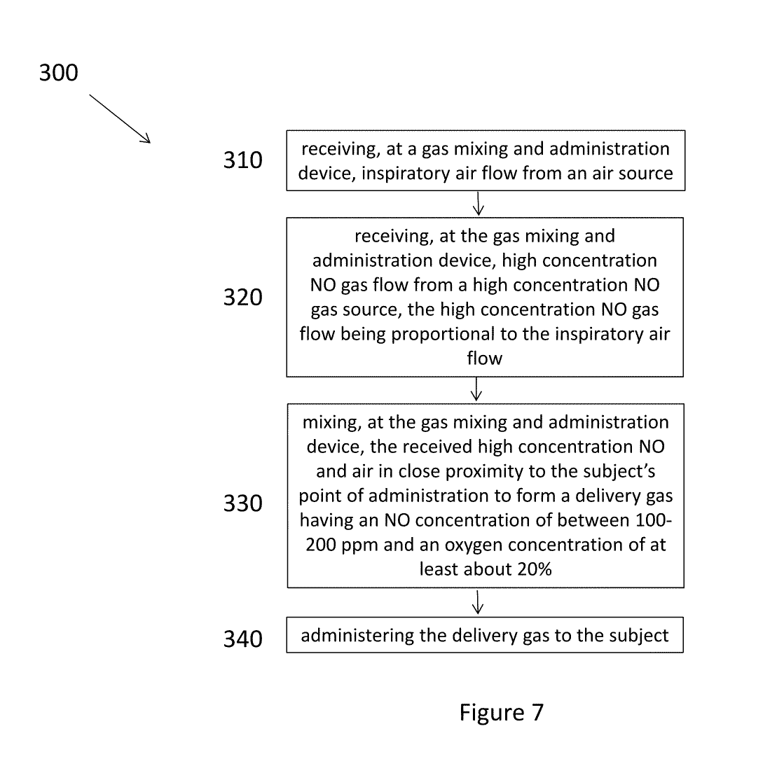

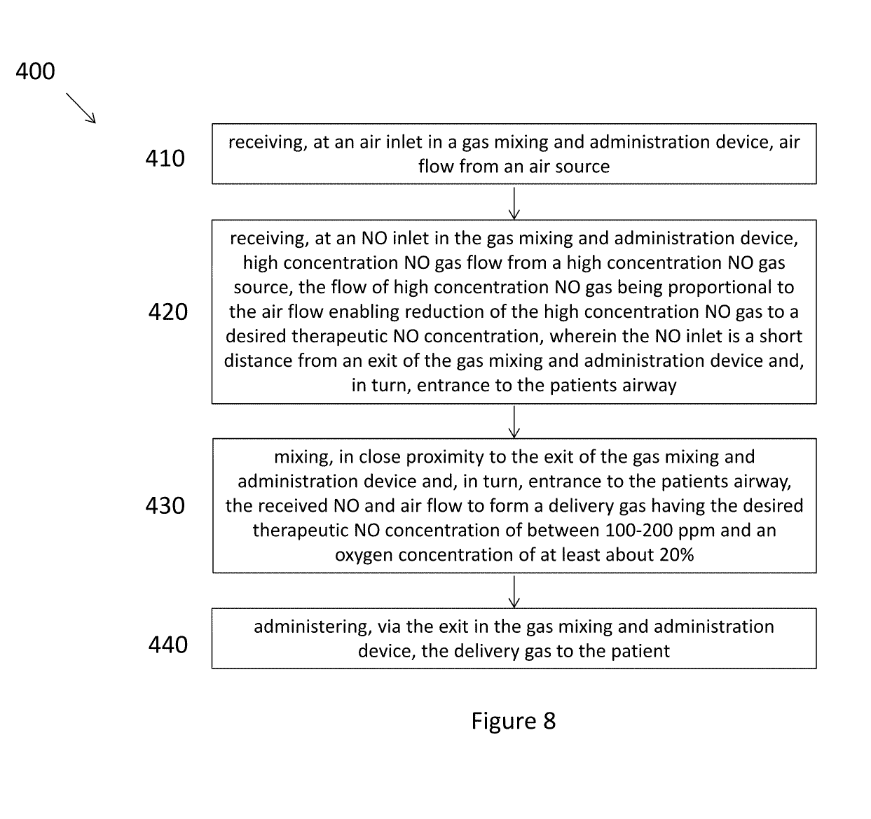

For example, as shown in FIG. 6, the systems contemplated herein can utilize and/or implement high dose NO delivery method 200 that comprises at least some of the steps of transferring and/or receiving a controlled quantity of a high concentration NO gas from a NO gas source or gas metering device to a mixing and administration device, at step 210, transferring and/or received air from an air source into the mixing and administration device, at step 220, mixing the transferred and/or received NO and air to form a delivery gas in close proximity to the subject's point of administration, at step 230, and administering the delivery gas to the subject, at step 240. In another example and as shown in FIG. 7, the systems contemplated herein can utilize and/or implement high dose NO delivery method 300 that comprises at least some of the steps of receiving, at a gas mixing and administration device, inspiratory air flow from an air source, at step 310, receiving, at the gas mixing and administration device, high concentration NO gas flow from a high concentration NO gas source, the high concentration NO gas flow being proportional to the inspiratory air flow, at step 320, mixing, at the gas mixing and administration device, the received high concentration NO and air in close proximity to the subject's point of administration to form a delivery gas having an NO concentration of between 100-200 ppm and an oxygen concentration of at least about 20%, at step 330, and administering the delivery gas to a patient, at step 340. In yet another example and as shown in FIG. 8, the systems contemplated herein can utilize and/or implement high dose NO delivery method 400 that comprises at least some of the steps of receiving, at an air inlet in a gas mixing and administration device, air flow from an air source, at step 410, receiving, at an NO inlet in the gas mixing and administration device, high concentration NO gas flow from a high concentration NO gas source, the flow of high concentration NO gas being proportional to the air flow enabling reduction of the high concentration NO gas to a desired therapeutic NO concentration, wherein the NO inlet is a short distance from an exit of the gas mixing and administration device and, in turn, entrance to the patients airway, at step 420, mixing, in close proximity to the exit of the gas mixing and administration device and, in turn, entrance to the patients airway, the received NO and air flow to form a delivery gas having the desired therapeutic NO concentration of between 100-200 ppm and an oxygen concentration of at least about 20%, at step 430, and administering, via the exit in the gas mixing and administration device, the delivery gas to the patient, at step 440.

In some embodiments, the delivery gas has a NO concentration in the range of about 100 to 200 ppm and an oxygen concentration of at least about 20%. In such an embodiment, the delivery gas is administered to a patient within a relatively short amount of time after the mixing of NO gas and air occurs, such that delivery gas has a low concentration of NO₂, for example a NO₂ concentration of less than 5 ppm. In at least one embodiment, the NO gas source used has a relatively high concentration of NO, for example 3,000 to 10,000 ppm. The use of high concentration NO as a NO gas source can eliminate the need to supply supplemental oxygen to the delivery gas, thus decreasing the risk of forming NO₂.

In various embodiments, the present invention includes steps and/or elements for using a metering device and mixing and administration device to control and/or maintain a NO concentration in the delivery gas in a predetermined range, such as in the range of 100 to 200 ppm. In at least one embodiment, the present invention can deliver NO in an intermittent dose form to the patient, wherein NO is only added to the delivery gas when the patient takes a breath.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out exemplary embodiments of the present invention, and are not to be construed as limiting in any way the remainder of the disclosure.

### Example 1

### Nitric Oxide Delivery at 160 ppm Using 5,000 ppm NO Source

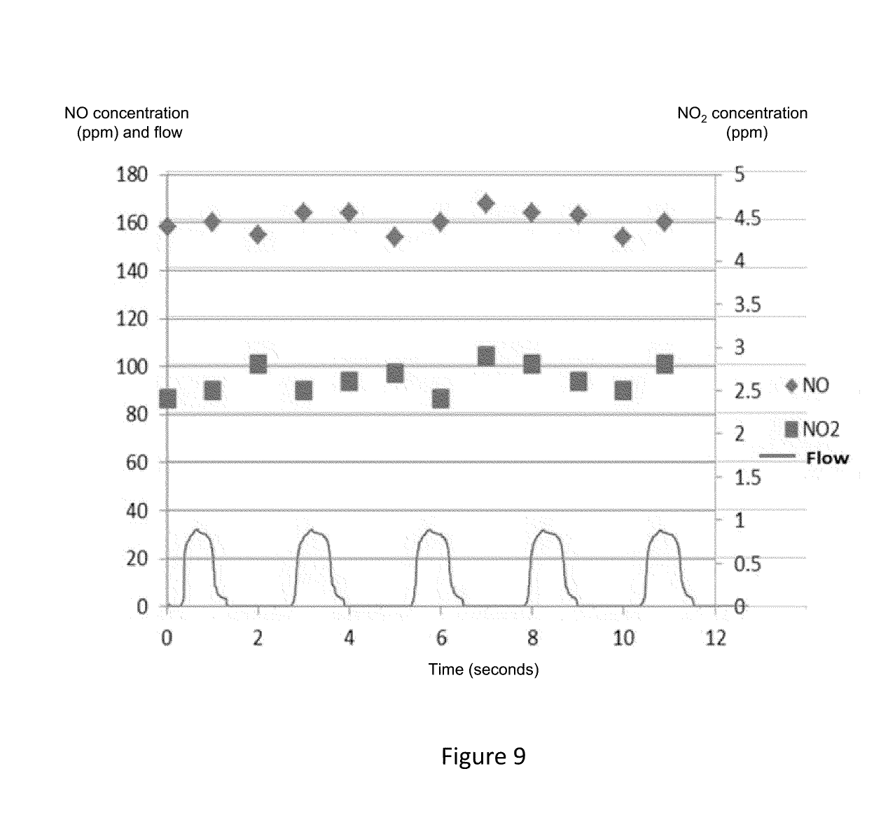

A stock cylinder containing 5000 ppm nitric oxide gas (Praxair, Connecticut) with the balance being nitrogen gas was connected to a dynamic gas metering system (12th Man Technologies, California) that was programmed to deliver a fixed concentration of 160±16 ppm NO. The metering system was connected to the gas mixing and administration device 140 as described in FIG. 2 and outlet 145 was connected to a mechanical test lung (Michigan Instruments, Michigan) that was configured to spontaneously breathe. A calibrated electronic flow sensor (TSI, Minnesota) was placed in-line with the metering system flow sensor and the output was connected to a computerized digital data collection system (LabView, National Instruments, Texas). The metering system contained sensors for monitoring inhaled NO, NO₂ and oxygen. Once the test lung started breathing simulation, continuous recording of the flow pattern, and measurement of the inhaled gas values in one second intervals were recorded. FIG. 9 shows representative data from several breaths recorded.

With the varying flow pattern of each inspiration, the metering system injected nitric oxide to maintain a constant concentration of 160 ppm within the targeted range. Measured NO₂ at all times was less than 5 ppm, with the measure NO₂ maintained at about 3 ppm or less. Systems currently known in the art are associated with NO₂ concentrations of about 4 ppm for NO delivery at 100 ppm, but such systems are associated with NO₂ concentrations of greater than 10 ppm for NO delivery at 160 ppm. Therefore the mixing and administration device of the present invention is able to demonstrate the ability to overcome a known problem for the delivery of relatively high concentrations of nitric oxide while maintaining acceptable NO₂.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method for administering nitric oxide (NO) gas to a patient, comprising:
receiving, at a gas mixing and administration device, a controlled quantity of high concentration NO gas flow from a high concentration NO gas source;
receiving, at the gas mixing and administration device, air flow from an air source into the gas mixer;
mixing, at the gas mixing and administration device, the received high concentration NO and air in close proximity to the subject's point of administration to form a delivery gas having an NO concentration of between 100-200 ppm and an oxygen concentration of at least about 20%; and
administering, via an exit in the gas mixing and administration device, the delivery gas to a patient.

2. The method of claim 1, wherein the received NO gas has a concentration in the range of about 3000 to 10,000 ppm.

3. The method of claim 2, wherein the received NO gas concentration is about 5000 ppm.

4. The method of claim 1, wherein the NO concentration of the delivery gas is about 160 ppm.

5. The method of claim 1, wherein the high concentration NO gas flow received from the high concentration NO gas source is from a gas metering device.

6. The method of claim 1, wherein the delivery gas is administered to the patient via a breathing tube connected to the gas mixing and administration device.

7. The method of claim 1, further comprising:
restricting, via a first one way valve, the delivery gas flow in the gas mixing and administration device to only forward flow in a direction towards the patient.

8. The method of claim 1, further comprising:
restricting, via a first one way valve, patient expiratory flow from entering the gas mixing and administration device.

9. The method of claim 1, wherein the air source is comprises atmospheric air.

10. The method of claim 7, wherein the atmospheric air has an oxygen concentration of about 21%.

11. The method of claim 1, wherein the delivery gas is formed without adding supplemental oxygen.

12. The method of claim 1, wherein the nitrogen dioxide (NO₂) concentration of the delivery gas is less than 5 ppm.

13. The method of claim 1, wherein the NO₂ concentration of the delivery gas is less than 3 ppm.

14. A method for administering nitric oxide (NO) gas to a patient, comprising:
receiving, at a gas mixing and administration device, inspiratory air flow from an air source;
receiving, at the gas mixing and administration device, high concentration NO gas flow from a high concentration NO gas source, the high concentration NO gas flow being proportional to the inspiratory air flow;
mixing, at the gas mixing and administration device, the received high concentration NO and air in close proximity to the subject's point of administration to form a delivery gas having an NO concentration of between 100-200 ppm and an oxygen concentration of at least about 20%; and
administering the delivery gas to a patient.

15. A method for administering inhaled therapeutic nitric oxide (NO) gas to a patient in need thereof, comprising:
receiving, at an air inlet in a gas mixing and administration device, air flow from an air source;
receiving, at an NO inlet in the gas mixing and administration device, high concentration NO gas flow from a high concentration NO gas source, the flow of high concentration NO gas being proportional to the air flow enabling reduction of the high concentration NO gas to a desired therapeutic NO concentration, wherein the NO inlet is a short distance from an exit of the gas mixing and administration device and, in turn, entrance to the patients airway;
mixing, in close proximity to the exit of the gas mixing and administration device and, in turn, entrance to the patients airway, the received NO and air flow to form a delivery gas having the desired therapeutic NO concentration of between 100-200 ppm and an oxygen concentration of at least about 20%; and
administering, via the exit in the gas mixing and administration device, the delivery gas to the patient.

16. A system for high-dose administration of nitric oxide gas to a patient, comprising:
a NO gas source having a NO concentration in the range of 3000 to 10,000 ppm;
a gas metering device, wherein the NO gas source is connected to the gas metering device via a first conduit;
a gas mixing device, wherein the gas metering device is connected to the gas mixing device via a second conduit, and wherein NO source gas from the NO gas source is transferred to the gas mixing device via the first conduit, gas metering device, and the second conduit;
an air source connected to the gas mixing device via a third conduit; and
a delivery gas dispensing mechanism for administering a mixed delivery gas to a patient.

17. The system of claim 12, wherein when the NO source gas is mixed with air in the mixing device, a delivery gas is formed having a NO concentration in the range of about 100 to 200 ppm.

18. The system of claim 13, wherein the delivery gas has an oxygen concentration of at least about 20%.

19. The system of claim 13, wherein the delivery gas has a NO₂ concentration of less than 3 ppm.

20. The system of claim 13, wherein the delivery gas has a NO₂ concentration of less than 5 ppm.

21. The system of claim 12, wherein the delivery gas dispensing mechanism is a conduit connected to the gas mixing device.

22. The system of claim 12, wherein the delivery gas dispensing mechanism is a breathing mask connected to the gas mixing device.

23. The system of claim 12, wherein NO concentration of the NO gas source is 5,000 ppm.
